(19) United States
(12) Patent Application Publication
(10) Pub. No.: US 2007/0094303 A1
(43) Pub. Date: Apr. 26, 2007

(54) System for management of processed instruments

(76) Inventors: Arthur Zwingenberger, Luzem (CH); Dorin Cioraca, Toronto (CA); Gabriel Neagoe, Toronto (CA); Robert Schroen, Toronto (CA); Robert Biermann, Toronto (CA)

Correspondence Address: CLARK & BRODY, 1090 VERMONT AVENUE, NW, SUITE 250, WASHINGTON, DC, 20005, US

(21) Appl. No.: 10/576,238

(22) PCT Filed: Nov. 5, 2004
(86) PCT No.: PCT/US04/36772

§ 371 Date: Dec. 29, 2006

Related U.S. Application Data
(60) Provisional application No. 60/517,080, filed on Nov. 5, 2003.

(51) Int. Cl. G06F 7/00

(52) U.S. Cl. 707/104.1

(57) ABSTRACT

An automated system to identify, analyze and record information associated with disinfection and/or sterilization procedures. Individual processing and client devices communicate via a computer network. The client devices such as desktop computers terminals, hand-held devices and/or process devices such as sterilizers, autoclaves, washing and/or disinfection devices are distributed among a health care facility more specifically disinfection and/or sterilization facilities that require processing of surgical instruments. The network server exchanges a variety of information with client devices as well as disinfection and/or sterilization equipment. In response to information from equipment and/or client devices, the network server may trigger decision messages to equipment and/or client devices, as well as generate reports for record keeping or maintenance purposes. The information distributed by the network server may pertain to the operation, maintenance and control of disinfection and sterilization equipment, characteristics and capabilities of instruments, and expert technical advice. The information received from the health care facilities includes requests for information, and process information documenting procedures performed within a health care, disinfection and/or sterilization facility.

### TECHNICAL FIELD

This invention relates to the art of processors for instruments, including sterilizers, disinfectors, washers, and the like. In particular the invention relates to a method and apparatus for tracking the contents of a container, such as a cassette used to contain articles or instruments placed in a processor and to validate the process to which the contents of the container have been subjected.

### BACKGROUND ART

It is known to utilize a cassette to hold articles to be subjected to sterilization. For example, medical instruments to be sterilized may be placed in a cassette and the cassette and the articles placed in a sterilizing chamber and subjected to a sterilizing procedure. Presently, an operator maintains a log of the articles that are placed in the cassette and the procedure to which the contents were subjected. That log may be maintained in a computer or may be as simple as a paper record.

A problem with the present procedure is that it relies heavily on the efficiency of the operator to record the articles placed in or removed from the cassette and maintain the physical integrity of the records. For example, if the cassette is to be maintained for an extended period of time after sterilization without opening it, the records must be properly maintained in a secure location. As well, the records must be updated when the cassette is opened and the instruments removed or replaced.

Acceptable sterilization processes also require a mechanism by which the process can be verified. Thus, it is often necessary to provide a system that records selected conditions to which the articles to be sterilized have been subjected. This is presently accomplished by, for example, a chemical “steam indicator,” which can be used only once and provides only a visual indication. When used with a containment vessel, such as a cassette, containing the articles, the cassette must be opened to be able to view the indicator. If the indicator shows that the sterilization has not been adequate, the containment vessel must be rejected and a new one obtained, which greatly disrupts the operation a results in a loss of time and resources. To minimize loss of time and disruption to the operation, it would be advantageous to be able to determine the status of the disinfection or sterilization load immediately after a disinfection or sterilization process but before the containment vessel is put away for storage or delivered to the point of use. Prior to the final processing of instruments for disinfection or sterilization it is essential to inspect the instruments for defects and/or maintenance requirements so that instruments not in acceptable condition are not made available to the end user. This is currently done by highly trained individuals and is relatively time consuming.

In a health care facility, disinfection and/or sterilization generally refers to the process of eliminating bacteria and other microorganisms from the surfaces of instruments, medical devices, implants and other articles used in surgical procedures. Sterilization connotes the absence of all life forms, including bacterial endospores, which are the living organisms most resistant to conventional sterilants. Disinfection, by distinction, only connotes the absence of pathogenic life forms (i.e., a bacterial endospore is not itself a pathogenic life form, but can produce such pathogens). Microbial decontamination is generic to both sterilization and disinfection.

A traditional sterilization process uses steam under pressure. Alternative sterilization processes use a chemical in the vapor phase as the sterilant. In each process, the sterilizer is designed to kill or reduce viable living organisms within a sterilization chamber. To achieve this objective, health care personnel must select the appropriate sterilization process and carefully monitor its parameters.

A traditional disinfection process uses a liquid chemical germicide. Automatic devices flush the liquid throughout the apparatus being disinfected at the correct temperature and for the correct duration of time. In each process, the disinfection device is designed to kill bacteria, viruses, mycobacteria and some spores within a chamber. To achieve this objective, health care personnel must select the appropriate process and carefully monitor its parameters.

To verify successful processing, health care facilities typically use chemical or biological indicators. A chemical indicator responds to one or more conditions necessary for proper processing, such as temperature, pressure, time, and sterilant concentration or exposure. A biological indicator carries a biological agent, and indicates successful sterilization when the biological agent has been killed. The indicator is placed on or within a pack containing articles to be disinfected and/or sterilized.

The indicator aids health care personnel in identifying packs that have been exposed to the conditions necessary for successful processing. The pack may carry other information, often within the indicator, that identifies the pack for record keeping purposes. For example, the indicator may carry text, a barcode, or a radio-frequency identification (RFID) tag with information that uniquely identifies the pack, and indicates status. In some cases, the information can be scanned in an automated manner to assist in automated record keeping via a computer system.

To achieve effective traceability, record keeping is essential in a health care facility and the facility must devote substantial personnel, training and administrative resources to the process. For example, it is necessary to maintain a sufficient inventory of sterilant, pack lists, and indicators, and properly maintain equipment. Comprehensive knowledge of procedures and control of associated parameters are necessary for proper disinfection and/or sterilization. In addition, efficient workflow requires effective tracking of packs to ensure that articles are available when needed. In addition, regulatory agencies and independent audit organizations may require access to records for verification of regulatory compliance or accreditation. Access to information concerning best practices also is important in maintaining and refining processes within a facility.

### SUMMARY OF THE INVENTION

In accordance with the invention, a container, such as a cassette, is provided with a tag, such as a radio frequency identification tag (RFID) that is capable of having recorded therein, as by a RF writer, the contents of the cassette and all other relevant information about the contents. As used herein, “container” should be construed to mean a container of a variety of sorts, including cassettes, boxes, covered trays etc.

For example, the RFID tag can be secured to the container such that it cannot become detached or it can be associated with the container in such other manner that its correlation with the container is secured. An operator would then use a known RF writer to record the contents of the container in the RFID when the container is loaded. Alternatively, each of the articles to be placed in the container is provided with an identification tag, such as an RFID, and the container is provided with a reader that automatically reads the tag on the article and updates the tag on the container. Or, the sterilizer could be provided with the reader/writer that is capable of reading the information from the instruments and recording it on the container tag when the container is placed in the sterilizer.

After the container with the tag thereon is loaded with the instruments, it can be placed in a sterilizer. The sterilizer is preferably provided with an automatic reader/writer that communicates with the RFID when the container is placed in the sterilizer and records such information as the date and time of sterilization and the sterilization protocol to which the container and its contents have been subjected. This can be scheduled in any of several ways, for example, by coordinating the reader/writer with the operation of the door to the sterilizer. In this example, the reader/writer is activated when the door is first closed and also when it is subsequently opened.

In one aspect, the invention comprises a combination of passive and active radio frequency identification systems, including an antenna and a power source enclosed in a housing and connected to digital temperature and pressure sensors, which are exposed to the environmental conditions of the disinfection and/or sterilization process.

When the entire device is to be enclosed in a containment vessel and placed in a disinfection and/or sterilization chamber, the housing is made of a material that withstands the environmental conditions of the sterilizing chamber. An antenna for communicating with external devices may be incorporated into the wall of the housing, and the digital temperature and pressure sensors are connected to the antenna and to the exterior of the housing. The temperature and pressure sensors may also be electrically connected through the housing wall to a monitoring system.

When the temperature and/or pressure of the disinfection and/or sterilization chamber exceed a preset temperature, the system becomes active and records temperature and pressure at preset intervals. When the temperature drops below the preset temperature, the recording stops, and the system switches to passive mode and waits until accessed by an authorized device that will analyze the data to determine, for example a pass or fail condition.

In another aspect, the invention is directed to an automated system and device for recording information and transmitting it to a central location or storage device in a format usable by a central management system. The invention may be implemented via a computer network, for example, having a network server and one or more client computers distributed among a number of process facilities. In particular, a unit in the network exchanges information with process facilities via the network. For example, a network server may provide the process facilities with information relating to device loads (e.g., a cassette and the instruments therein) or processes. In response to information received from the process facilities and/or devices, the network server may trigger distribution of materials to the process facilities, and generate reports for record keeping purposes.

In accordance with other aspects of the invention, the container having a tag thereon that records the contents of the containers is removed from the sterilizer after sterilization and stored. The sterilizer will automatically record on the tag the time of removal from the sterilizer and the contents of the container and the processes to which they have been subject and this information can then be read from the tag without opening the container.

The physical structure includes the tags and the reader/writer. The tag is preferably secured to the container as described or correlated with the container if not attached. The reader/writer on the sterilizer must be placed in such a location that it can read from and write to the tag on the container. As well, if the container is provided with a reader/writer for communicating with the tags on the individual instruments, it must be appropriately placed to allow such communication. Because containers are typically metal, the container may be provided a small window of transparent material to allow such communications.

### BRIEF DESCRIPTION OF THE DRAWINGS

FIG. 1 is a block diagram of a multi-facility health care network summarizing key areas involved in the disinfection, sterilization and use of surgical instruments.

FIG. 2 is a block diagram of a health care facility summarizing key areas within the facility involved in the disinfection, sterilization and use of surgical instruments.

FIG. 3 is a block diagram of the flow of instruments through a health care facility.

FIG. 4 is a block diagram of the components of a disinfection and/or sterilization process indicator according to the invention.

FIG. 5 is a perspective view of a cassette, having a bottom tray and upper lid, that forms a disinfection and/or sterilization vessel for medical and/or dental instruments with installed sensors, power supply, central processing unit and an antenna.

### DETAILED DESCRIPTION OF THE PREFERRED EMBODIMENTS

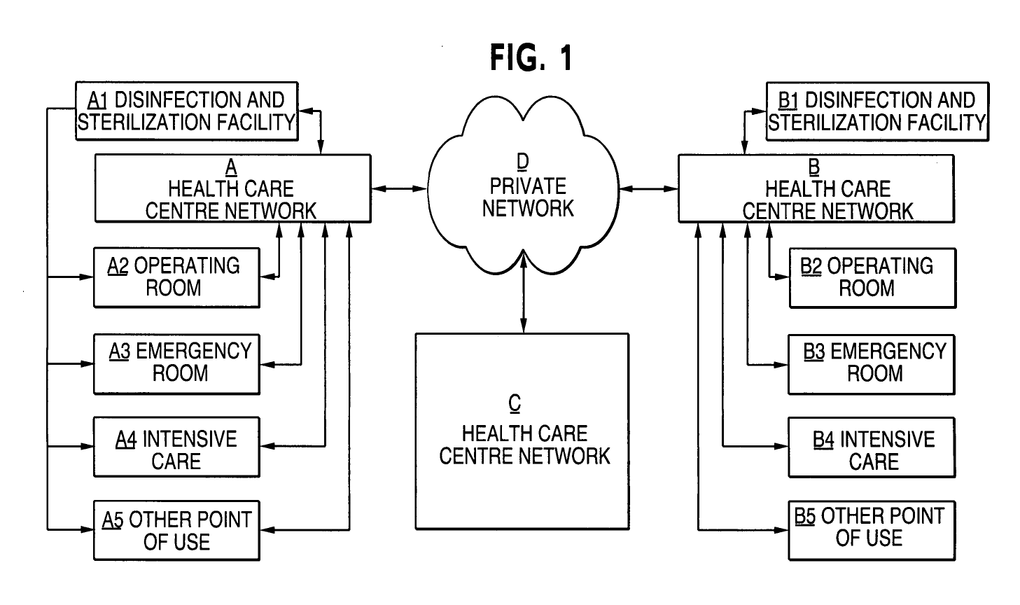

Two or more health care centers A-B as shown in FIG. 1 are capable of sharing and inter-linking records via a private network D and optionally centralize all records at a central network server C. Each health care center consists of Disinfection and Sterilization Facility A1 and B1 providing services to several point-of-use areas within the center such as operating rooms A2 and B2, emergency rooms A3 and B3, intensive care units A4 and B4 as well as other specialized care areas A5 and B5. By providing a time effective method of capturing information relating to the process and use of instruments though a health care center and inter-linking all of the points of contact it is possible to retrieve information for statistical, quality and/or recall purposes in a significantly reduced amount of time than currently common in many health care centers.

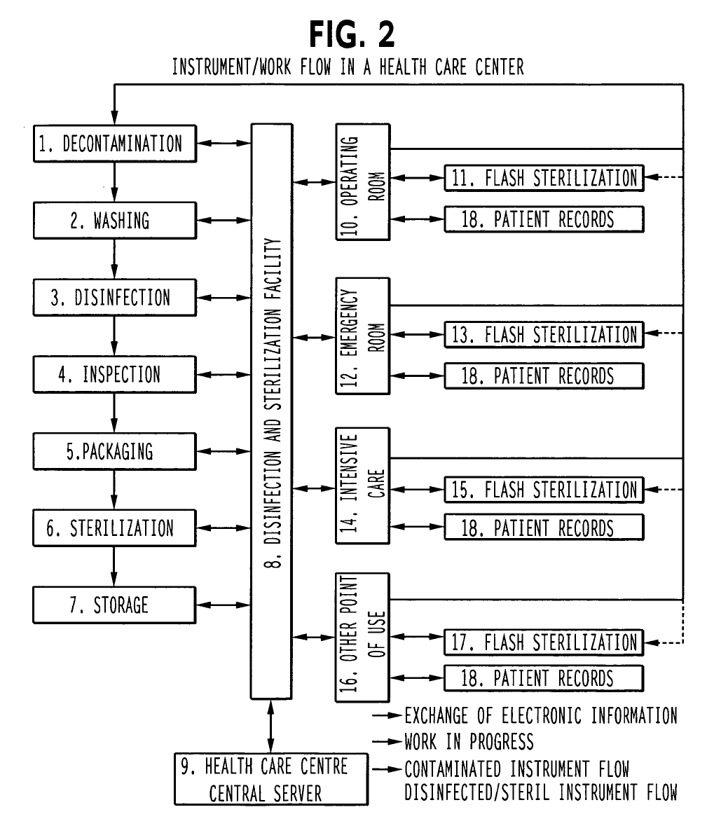

In FIG. 2 the block diagram emphasizes and shows a summary of contact points starting with the functions normally associated with the disinfection and sterilization facility 8 that deals with a specific order of events including 1. Decontamination, 2. Washing (step 1 and 2 is only required if the instruments have been used), 3. Disinfection, 4. Inspection, 5. Packaging, and 6. Sterilization. If the instruments are not required immediately they are stored for later distribution otherwise they are issued to point-of-use areas. At each of the above mentioned steps it is important to determine if each instrument is properly processed for functionality and location control, therefore at each step relevant information is electronically captured and transmitted to a central data base server. Once the instruments arrive at the point-of-use areas such as an operating room 10, emergency room 12, Intensive care or other areas 16, the container and/or the instruments are scanned and linked to the patient file 18, should any of the instrument(s) require to be “flash” sterilized 11, 13, 15, and/or 17 all transaction and result information is also recorded and stored in a central database 9.

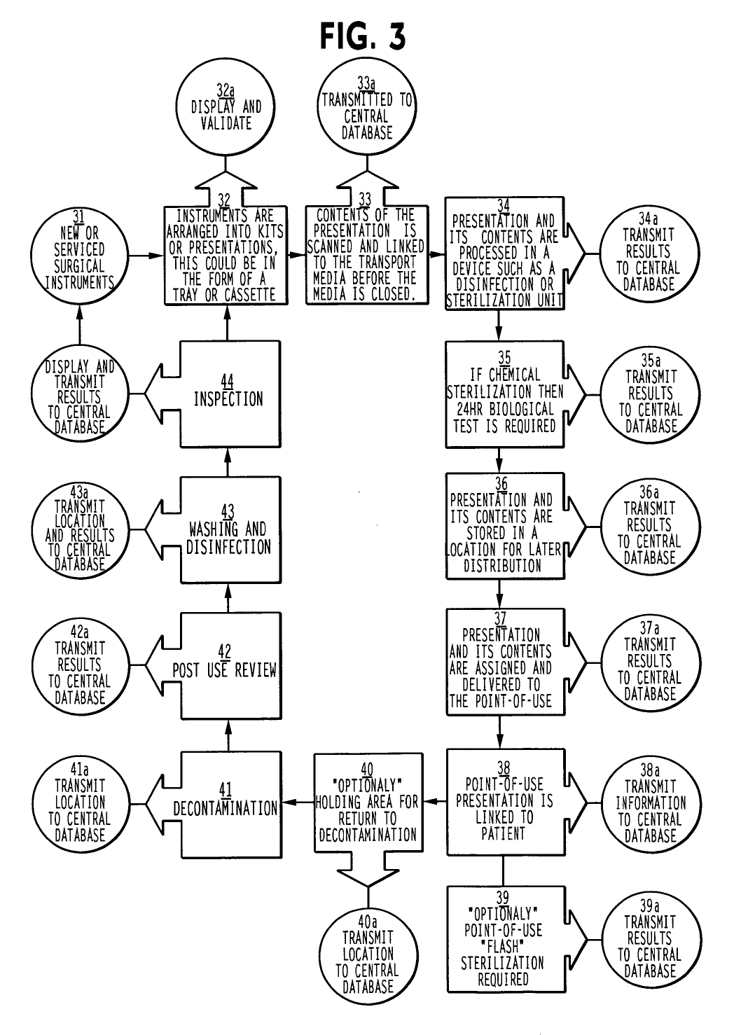

FIG. 3 represents a detailed block diagram showing each point of contact of a surgical instrument through a health care facility. The process starts with the acquisition of a new or return of a refurbished surgical instrument 31, at this point the instruments are inspected and verified to required specifications. It is assumed that the instruments are in a clean disinfected state and optionally have a form of marking to identify the instrument by means of a machine-readable barcode and/or radio frequency identification (RFID) tag.

Depending on the uniqueness of the instrument it may be put into storage or immediately assembled and arranged 32 into a surgical presentation commonly referred to a kit or cassette. During the assembly stage the operator has access to a computer display 32a, to guide them through a visual representation of the layout of the instruments, with an optional machine-reading device to identify the instruments being handled it is possible to provide visual and/or audible indications if an instrument is being placed that is not called for. The machine-vision device described in the inspection process 44 can also be used to determine correct positioning of the instruments into the presentation. The final stage of arranging the presentation is to include a “process indicator” as described in claim 13. The instruments can be scanned in though a stage 33 utilizing barcode and/or radio frequency identification (RFID) tags gathering information resulting in a transmission 33a to the central facility or health care database server. If the instruments are placed in a metallic container and the instruments are equipped with radio frequency identification tags this operation would have to be done before the container is closed otherwise the presentation would be wrapped with surgical towels and a form of “Surgical Wrap” allowing radio frequency identification tags to read through the material. At this point a link is developed between the instrument, presentation and optionally the identification of the assembler of the presentation, this information including location is transmitted 33a to the central facility or health care database server.

The presentations are then processed through a method of disinfection and/or sterilization 34. Utilizing the radio frequency identification tags attached to the presentation the processing device can automatically start a process based on the presentations and/or its contents and validate the process if the operator manually changes or overrides the process. Process characteristics are recorded and linked to the contents of the process load and transmitted 34a to the central facility or health care database server.

Since there are several methods of disinfection and/or sterilization some process will require specific process validation, this could a simple process indicator and/or detailed biological testing 35 that would require for the load to be quarantined until the results of the tests are confirmed. Under these circumstances the processing device has the ability to “write” to instruments and/or presentations equipped with radio frequency identification tags information relating to the quarantine period as well as transmit 35a resulting data to the central facility or health care database server.

After the presentation(s) including its contents instruments have been cleared the required quarantine period (if applicable) the presentation can be stored 36 or assigned for immediate distribution 37 to a point-of-use area such as an operation room, emergency room, intensive care or other point-of-use area. Utilizing network client devices equipped to read radio frequency identification tags or barcodes the location of the presentations is transmitted 36a and 37a to the central processing facility or health care database server.

When the presentation is put to use 38 the utilization of network client devices equipped to read radio frequency identification tags or barcodes can link the presentation to the health care individual and/or teams as well as the patient, transmitting 38a information to the central processing facility or health care database server. If required the instruments may be require to be “flash sterilized” 39 where the device could transmit 39a identification of the instrument, process device, and result to the central processing facility or health care database server.

With the machine-readable identification of the presentation and instruments it is possible to validate that all instruments are accounted for insuring that they are not misplaced, lost or inadvertently remained in a surgical cavity transmitting the conclusions to the central processing facility database server.

If required the soiled and/or unused instruments may be accumulated in a “holding” area 40 prior to return to decontamination where the location of the instruments are transmitted 40a to the central processing facility database server.

During decontamination 41 the instruments are cleaned organized and location controlled, the presence of machine-readable markings such as barcodes and/or radio frequency identification tags will assist in recording and process requirements by transmitting 41a collected data to the central processing facility database server.

Generally a post-use-review 42 is done to identify missing, damaged and/or worn out instruments, this information is transmitted 42a to the central processing facility database server.

During an automated washing and disinfection process 43 the instruments can be identified as to process requirements, some process devices have the ability to optionally condition the instruments such as immersing them in a water-soluble lubricant solution to lubricate hinges and/or locks, this process is known as “milking”, the fact the instruments have gone through the disinfection state and have been subjected to conditioning can be transmitted 43a to the central processing facility database.

Inspection 44 can be done manually or more accurately utilizing a machine-vision device where the process device can identify an instrument using high definition cameras to read a barcode on the instrument or read a radio frequency identification tag to request specification information which will allow the machine-vision device to analyze the instrument to be inspected. The result of the inspection can be transmitted 44a to the central processing facility database server as well as notify the operator visually and/or by auditory means for pass or fail condition of the instrument. Should the instrument fail the inspection the operator would take appropriate steps to dispose of the instruments or prepare the instrument for service, either direction would be transmitted 44a to the central processing facility database to maintain the condition and/or location of the instrument.

As a result of the flow shown in FIG. 3 a comprehensive electronic link can be established between: instrument, presentation, operator, process device, process, location, point-of-use, surgical procedure, operating team and/or individual, and patient records facilitation a fast and efficient traceability of key point that may be required to be retrieved in the case of a follow-up or recall situation.

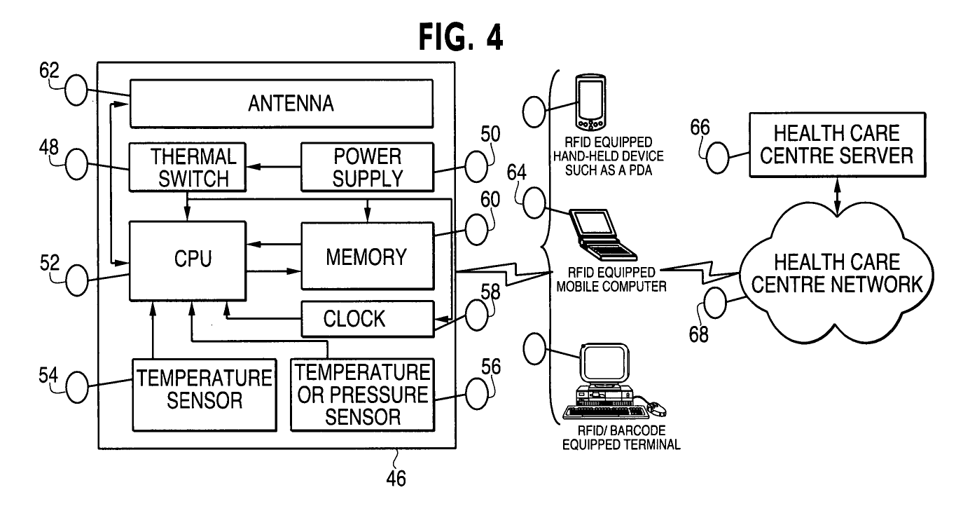

A disinfection and/or sterilization process indicator device 46 as show in FIG. 4 consists of eight basic components; a thermal switch 48 that will activate the power supply 50 at a preset temperature and/or pressure and thus execute a program within the central processing unit (CPU) 52 to record temperature readings from sensor 54, pressure reading from sensor 56, and elapsed time from the internal clock 58 to storage memory 60. After the device has recorded all relevant information the device switches to a passive mode until it receives a signal from an external device 64 equipped with radio frequency identification (RFID) hardware and software via the built-in antenna 62. The external devices 64 can be in a form of personal computers and/or hand-help devices and will analyze the data and determine a pass or fail condition and optionally transmitting a clear command to the process indicator device and/or to a central health center server 66 for archival purposes via a private network 68.

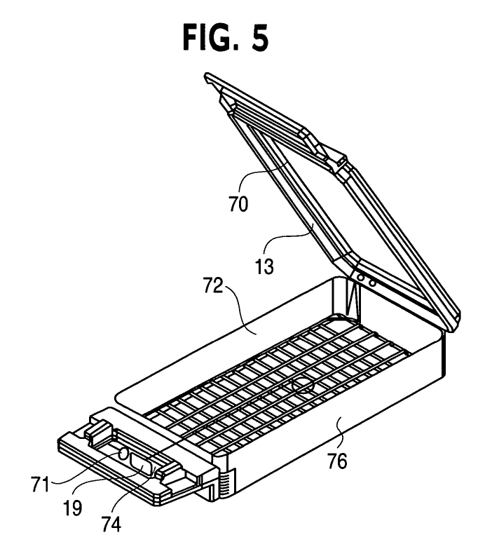

The process indicator device can also be an integral part of the sterile containment media employing the same principles as described for FIG. 4, a disinfection and/or sterilization vessel generally in the form of a rectangular cassette, designed to be placed into a disinfection and/or sterilization apparatus (not shown), FIG. 5 shows one form of several adaptations of a cassette currently on the market. The cassette includes an upper lid 70 and a bottom tray 72, which could be joined with one another by a hinge. The electronics on the device would be place in a convenient area that would allow the accessibility of the power supply 71 and antenna 19 without opening the cassette. The temperature and pressure sensors 76 are shown to be placed in the center of the cassette. As well, the cassette may be provided with a window to allow reading tags on elements within the container.

The process indicating tag may be placed in a variety of places, including the seal between the lid and tray. This seal may also include a tag indicating the number of cycles to which it has been subjected to verify that it is within its lifetime.

In accordance with the general concepts of the invention, instruments used in a health care facility such an operating room, emergency room or other point-of-use areas could have a barcode and/or a radio frequency identification (RFID) tag known as “machine-readable” are embedded or attached to them. Acquisition information such as date, vendor and/or property number can be included in the machine-tags, and instruments can be organized and “kitted”, into surgical presentations such as a tray or a cassette, which could also be identified with barcodes and/or radio frequency identification tags or they could be individually wrapped.

A machine-readable operator ID can be recorded by a reader and written back to presentations and/or instruments if they are equipped to do so for record keeping purposes. By automatically reading the machine-readable markings, if an instrument is included into the presentation that is not called for, a visual and/or audible warning could notify the operator. The surgical presentation can be identified by means of a barcode and/or a radio frequency identification (RFID) tag to link the instruments to the presentation.

Process information such as the operator ID could be written back to instruments and/or presentations if they are equipped with radio frequency identification (RFID) tags for traceability purposes.

The radio frequency identification, (RFID) capable process indicator would make it possible to read the results through the “Sterilization Wrap” that surgical presentations are normally enclosed in without the need of a direct line of sight or depending a human interpretation if the process passed or failed.

The presentation and its contents are then processed by means of a device utilizing a disinfection and/or sterilization method of choice (for example steam, gas or chemical).

Optionally the system could include a means for disabling the process device if the contents to be processed are not compatible with the selected process of the device.

Optionally the system could include a means to automatically initiate the appropriate process for execution based on the contents to be processed.

Optionally the system could include a means to disable the device if components are used that could interfere with the proper function of the device, such as filters, seals, gaskets etc.

The device is capable of retaining information from each process or load it executes, wherein the information may include processing characteristics for the individual load, the processing characteristics for each load including at least one of; type of device, device identification, cycle time, process time, temperature, pressure, humidity and/or chemical concentration.

The device is capable of reading information from instruments and/or presentations utilizing a built-in reader such as a barcode and/or a radio frequency identification (RFID) readers.

The device could write back information regarding the last process to the instruments and/or presentations that are equipped with radio frequency identification (RFID) tags relating to what they have been exposed to.

The device could produce a printed document that would include process information such as load number, process type, process characteristics etc. as stated in claim 19.

The disinfection and/or sterilization device used in the process of disinfecting and/or sterilization can be connected to an Ethernet/Internet backbone utilizing an Internet Protocol (IP) for communication to other devices such as central database servers and hand-held computers.

The disinfection and/or sterilization device could be administered via an internet browser utilizing network clients such as hand-held computers.

The disinfection and/or sterilization device could have user ID and password restrictions.

The disinfection and/or sterilization device could receive information from a central health care server, service organization and/or manufacturer relating to updates and/or maintenance requirements which it could be displayed to users at the device and/or via a internet browser as a WEB page on computer terminal or hand-held computer.

The disinfection and/or sterilization device could transmit information relating to its condition to a central server in order to trigger a demand for maintenance and/or technical review.

The disinfection and/or sterilization device is linked to the process it has executed which is in turn linked to the “surgical presentation” which could have been linked to the instruments if the instruments are marked with machine-readable identification methods such as radio frequency identification (RFID) tags or barcodes.

The device when connected to an Ethernet/Internet backbone utilizing an Internet Protocol (IP) has the ability to transmit collected data to a destination of choice such as a central database server in a health care facility.

The format of the information could be in a format (such as Hypertext Markup Language (HTML), Dynamic Hypertext Mark-Up Language (DHTML, or Extensible Mark Up Language (XML)) that could be used by a central system.

The central health care server could then generate reports, which includes the integrated process information, presentation identification and/or instrument identification received from one or more of the disinfection, sterilization devices and/or other process devices within the facility or facilities.

If the process devices are dependant on a consumable material such as a sterilant and/or indicators the device could include the identification of the material and consumption amount in the transmission to the central database server.

If the presentation includes a radio frequency identification (RFID) capable process indicator the scan of the device could pass or fail the presentation before making it to the point-of-use area, which would otherwise result in significant time loss.

The device could write back information regarding the last process to the radio frequency identification (RFID) capable process indicator.

The surgical presentation could be stored for later use or immediately assigned to a point-of-use area.

The location of the instruments and/or the presentations could be recorded and transmitted via independent network clients that are equipped with barcode and/or radio frequency identification (RFID) readers to a central health care database server.

The network server provides access to the data collected by other network clients and/or devices to a network client via the computer network to generate a report and/or display of resulting data for purposes of process validation and/or location of instruments and/or presentations.

Upon demand the surgical presentation is moved to the point-of-use when it can be linked to a patient file.

The patient file could be linked to the procedure and surgical team by means of a barcode and/or radio frequency identification (RFID) tag attached to the patient record.

If an instruments sterility inadvertently becomes compromised it would be required to be disinfected and/or sterilized before use, this could be done by requesting a replacement, returning the instrument to the disinfection and sterilization facility, this would incur a significant time loss. A common method to deal with situation of this kind is to “flash sterilize” the instrument in the point-of-use area.

The combination of identification methods (barcode/radio frequency identification (RFID)) could be used to validate the location of the instruments

Remote devices such as hand-held computers, notebooks and/or computer terminals equipped with scanning devices could be used to collect and transmit information as to their location and/or availability.

Optionally entire trolleys, which contain soiled instruments and/or presentations are processed via a walk-in decontamination washer.

Post-use-review of presentations and/or instruments are “surface cleaned” and re-organized.

Instruments are washed (high temperature disinfection) and optionally lubricated (known as “milking”), process devices which are equipped with the ability to read machine-readable markings, could automatically process required steps and/or transmit results to a central health care database server.

Instruments are moved to the pre-sterilization process and location of the instruments can be recorded and transmitted by designated client devices to the central database server.

A machine-vision device could be used to interact with and identify the instruments as they travel to the presentation assembly area.

Based on the identification the system could retrieve specific characteristics of the instruments to analyze and pass or reject the instrument by notifying the operator by visual or audible indicator via a computer display.

All the information relating to usage of materials; information relating to processes; identification of presentations and/or instruments could be provided as an interactive communication between technical personnel knowledgeable in the processes and facility personnel and the disinfection and/or sterilization devices.

With the ability to record and store information in central database that comprises of process, process device, process results and item details that link to each other, it is also possible to trace each item which has been used and therefore be able scrutinize results from archival storage. Further, it is possible to trace each patient that received, and/or was treated and/or was in contact with any of the above in the event that notification or follow up is required.

Modifications within the scope of the appended claims will be apparent to those of skill in the art.

1. A method comprising:
collecting process information relating to processing content from an indicator on a device used in a washing, disinfection or sterilization facility via a computer network;
storing the information electronically to allow for the generation of reports or displays based on the received information; and
providing a network client with access via the computer network.

2. The method of claim 1, further comprising transmitting collected process information from the device used in a the disinfection or sterilization facility via the computer network; to subsequently store electronically to allow for the generation of reports or displays based on the received information; and providing the network client with access via the computer network.

3. The method of claim 1, wherein the network client is associated with a reviewer that analyzes that data to evaluate compliance with washing, disinfection or sterilization processing standards.

4. The method of claim 3, wherein the network client includes authorized facility staff members, manufacture support groups, regulatory agency or an audit organization.

5. (canceled)

6. The method of claim 1, further comprising the steps of providing a cassette for storing said device, said cassette having an electronically readable cassette tag, storing information regarding said device on said cassette tag, and placing said device in said cassette.

7. The method according to claim 6 wherein said step of storing information regarding said device comprises optically scanning a code on said device.

8. The method according to claim 6 wherein said step of storing information regarding said device comprises electronically reading an instrument tag on said instrument.

9. The method of claim 6 further comprising establishing a link between the plurality of contact points to allow retrieval of the stored information.

10. The method of claim 6, wherein the step of collecting includes identifying an instrument as the device.

11. A method according to claim 1 further comprising:
electronically reading status information from an indicator at a plurality of contact points relating to a sterilization or disinfection condition of an instrument; and
storing the information electronically on one or more RFID tags.

12. An RFID tag configured to receive, record, store and make available for subsequent electronic transmission information relating to an instrument to be washed, disinfected or sterilized or to a container configured to receive said instrument.

13. An RFID tag according to claim 12 wherein said information comprises at least one of the following:
the model number of said instrument or container,
the lot number of said instrument or container,
the serial number of said instrument or container,
the type of process to which said instrument or container has been subjected,
the date and time of one or more processes to which said instrument or container has been subjected,
the status of a process to which said instrument or said container is subjected during such process,
the number of processes to which said instrument or container has been subjected,
the identify of an operator initiating or responsible for a sterilizing or disinfecting process to which said instrument or container has been subjected,
the service history, owner, or location of said container,
the revision number of said instrument or container,
the part number of said instrument or container,
the reorder number of said instrument or container,
the expected life of said instrument or container,
the date of manufacture of said instrument or container,
the date of installation of said instrument or container,
the date of first use of said instrument or container, or
the electronic product code (EPC) of said instrument or container.

14. A tag according to claim 12 in combination with said container for a plurality of said instruments.

15. A tag according to claim 12 in combination with said container comprising:
a tray for receiving said plurality of instruments to be washed, sterilized or disinfected,
a lid for covering said tray and forming an enclosure, and
a seal between said tray and lid for sealing said enclosure and having at least a portion of said tag embedded therein.

16. A tag according to claim 12 in combination with a seal configured to fit between a tray and a lid of said container.

17. A tag according to claim 16 at least partially embedded in said seal.

18. A tag according to claim 17 wherein said tag is capable of accumulating the number of washing, sterilizing or disinfecting procedures in which said seal has been used.

19. A tag according to claim 12 in combination with apparatus for sensing and indicating a washing, sterilization or disinfection process to which an instrument has been subjected comprising:
a power supply,
a switch for determining when a predetermined temperature and/or pressure condition has been reached and for activating said power supply in response thereto,
a sensor for detecting sterilization or disinfection temperature and/or pressure conditions at a contact point, and
a central processing unit for processing the information from the sensors and writing said information to said RFID tag for subsequent retrieval.
